(19) United States
(12) Patent Application Publication
(10) Pub. No.: US 2023/0012788 A1
(43) Pub. Date: Jan. 19, 2023

(54) SUBSTANCE OVERDOSE DETECTION AND TREATMENT DEVICE

(71) Applicant: Hamish Millar, North Vancouver (CA)

(72) Inventor: Hamish Millar, North Vancouver (CA)

(21) Appl. No.: 17/305,821

(22) Filed: Jul. 15, 2021

(51) Int. Cl. A61M 5/172; A61M 5/145; A61M 5/158

(52) U.S. Cl. CPC A61M 5/1723; A61M 5/1452; A61M 5/158; A61M 2005/1585; A61M 2005/1726; A61M 2205/18; A61M 2209/088; A61M 2205/3576; A61M 2230/205; A61B 5/4845

(57) ABSTRACT

A drug overdose detection apparatus that is configured to measure the blood oxygen level of a user and upon detection in a drop thereof provide administration of a substance to counteract the drug overdose. The present invention includes a housing that is configured to be worn by a user has an interior volume. Disposed in the interior volume of the housing is a substance administration assembly wherein the substance administration assembly includes a syringe member, a plunger member and a plunger driver. A motor is operably coupled to the plunger driver and facilitates movement thereof. A spring needle assembly is operably coupled to the substance administration assembly and includes a spring biased needle that is operable to inject into a patient so as to inject the substance. The present invention further includes an audio alarm and transceiver configured to provide alerts and transmit signals.

### FIELD OF THE INVENTION

The present invention relates generally to healthcare devices, more specifically but not by way of limitation, a wearable monitoring device that is operable to measure at least one biometric parameter of a wearer in order to diagnose a potential overdose of a drug substance such as but not limited to opioids and initiate initial treatment thereof.

### BACKGROUND

Opioid's have been utilized for decades as a pain management drug for patients. This class of drug is inexpensive to manufacture and patient costs are relatively inexpensive. While there are commercial and health benefits to employing a pain management strategy with this drug class, there are serious challenge in the recreational realm of opioid use/abuse. Opioids and similar drugs while highly effective are also very addictive. Patients can become addicted to these drugs in as little as a week. As patients increase their utilization of these drugs there are inherent risks to their health.

An opioid epidemic has hit North America over the last decade and it has resulted in tens of thousands of deaths of patients many of which have resulted from an overdose. Most of these results from recreational, or non-monitored, use of opioids. Many of these deaths could have been prevented but monitoring of the patients being administered these drugs is challenging. In many areas the patients must submit to a monthly urine test wherein there urine specimen is tested to ensure the appropriate and/or prescribed amount of opioid is present. Outside of these types of test there are no other routine testing procedures. If an overdose occurs there are treatments that can help a patient survive such as but not limited to naloxone. However, if a patient is by themselves in an event of an overdose it is not likely that emergency medical care can be administered in a timely manner so as to potentially save the individuals life. It should be noted that the primary purpose of the invention disclosed herein is for the purpose of providing protection of an overdose for those individuals who intently use opioids as well as others who may fall into alternate categories of use. Another challenge with opioids is that this substance can be used to lace other recreational drug products in the heroin, fentanyl or other recreationally purchased pharmaceuticals and as such accidental overdoses are increasingly common.

Accordingly, there is a need for a patient monitoring device that can be worn by a person taking opioids, wherein the device can measure oxygen saturation and administer an overdose antidote in the event an overdose is detected automatically. It should be noted that the primary purpose of the invention disclosed herein is for the purpose of providing protection of an overdose for those individuals who intently use opioids as well as others who may fall into alternate categories of use.

### SUMMARY OF THE INVENTION

It is the object of the present invention to provide a wearable drug overdose detection apparatus that is operable to utilize biometric measurements to detect an overdose wherein the present invention in a preferred embodiment is encased in a housing that is releasably secured to a limb of a patient.

Another object of the present invention is to provide an overdose detection apparatus that is configured to automatically administer an antidote wherein the present invention includes a oxygen saturation measurement element.

A further object of the present invention is to provide a wearable drug overdose detection apparatus that is operable to utilize biometric measurements to detect an overdose wherein the apparatus further includes a central processing unit configured to provide operation of the present invention.

Still another object of the present invention is to provide an overdose detection apparatus that is configured to automatically administer an antidote wherein the present invention includes at least one syringe.

An additional object of the present invention is to provide a wearable drug overdose detection apparatus that is operable to utilize biometric measurements to detect an overdose wherein the at least one syringe has an antidote loaded therein and is operably coupled to a motor or is spring powered/motor released.

Yet a further object of the present invention is to provide a wearable drug overdose detection apparatus that is operable to utilize biometric measurements to detect an overdose wherein the at least one syringe includes a spring loaded needle operable to inject into a wearer.

Another object of the present invention is to provide a wearable drug overdose detection apparatus that is operable to utilize biometric measurements to detect an overdose wherein the motor or spring operably drives a syringe plunger.

Still an additional object of the present invention is to provide an overdose detection apparatus that is configured to automatically administer an antidote wherein the motor is further operably coupled to a lever switch to engage operation of the at least one syringe.

Still a further object of the present invention is to provide a wearable drug overdose detection apparatus that is operable to utilize biometric measurements wherein the present invention could further employ a servo style motor to operate the at least one syringe.

Yet an additional object of the present invention is to provide an overdose detection apparatus that is configured to automatically administer an antidote that is battery operated and includes status indication lights.

Still another object of the present invention is to provide a wearable drug overdose detection apparatus that is operable to utilize biometric measurements wherein the present invention can further include a transmitter to provide notification to emergency healthcare personnel.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is an exemplary diagrammatic view of the present invention; and

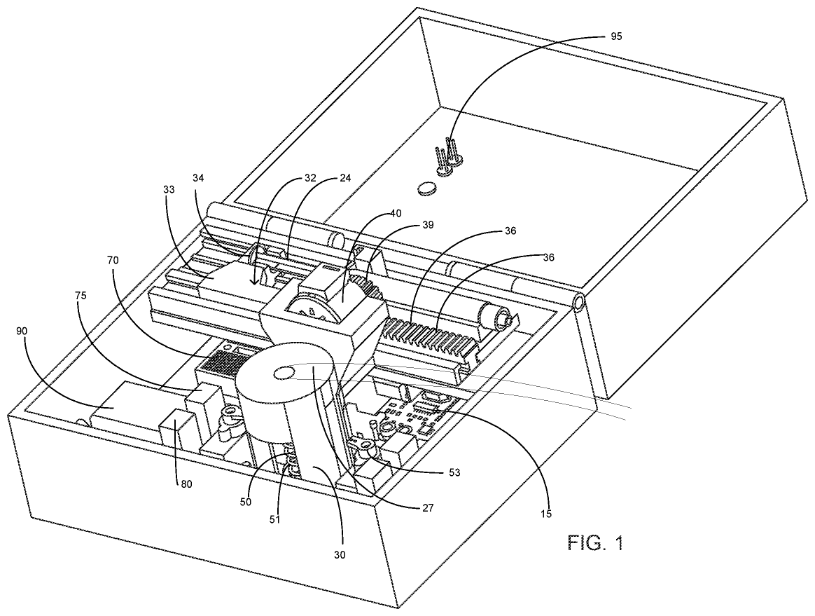

FIG. 2 is a side perspective view of an exemplary embodiment of the present invention.

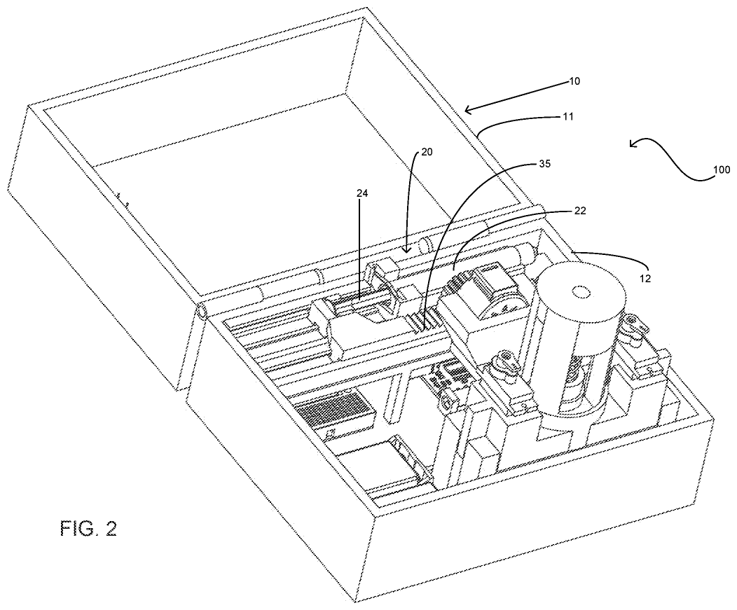

### DETAILED DESCRIPTION

Referring now to the drawings submitted herewith, wherein various elements depicted therein are not necessarily drawn to scale and wherein through the views and figures like elements are referenced with identical reference numerals, there is illustrated a substance overdose detection apparatus 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted as a part hereof, the substance overdose detection apparatus 100 includes a housing 10 wherein the housing 10 includes a first portion 11 and a second portion 12 that are hingedly secured so as to provide access to the interior volume of the housing 10. The housing 10 is manufactured from a durable rigid material such as but not limited to plastic. It should be understood within the scope of the present invention that the housing 10 illustrated herein is exemplary only. In the preferred embodiment of the present invention it is contemplated that the housing 10 be formed in a small non-intrusive form that is adapted to be worn on an arm, leg or other body part with minimal to no-obstruction of regular movements. The housing 10 could be releasably secured to an arm of a wearer utilizing suitable materials such as but not limited to straps. It should be further understood within the scope of the present invention that the housing 10 could be configured to be releasably secured to a belt or similar garment structure and wirelessly coupled to an oxygen saturation detector that is present on a finger or other body part of a user.

Disposed within the housing 10 are the operational components of the substance overdose detection apparatus 100. The substance overdose detection apparatus 100 is operable to measure oxygen saturation for a wearer and upon detection of a rapid drop therein. During an overdose of an opioid a person will experience a rapid drop in oxygen saturation in their blood stream. It is the intention of the present invention to establish a baseline oxygen saturation level for a wearer and upon detection of a rapid drop therein administer an antidote in order to render emergency aid to the wearer. All of the elements discussed hereinafter can be embodied in alternate forms as a result of contemplated designs of the present invention. The substance overdose detection apparatus 100 includes a central processing unit 15 that is operable to provide operation and control of the substance overdose detection apparatus 100. The central processing unit 15 is disposed within the interior volume of the housing 10 and includes the necessary electronics to receive, store, transmit and manipulate data. The central processing unit 15 is either directly or indirectly operably coupled to all of the elements of the substance overdose detection apparatus 100.

The substance overdose detection apparatus 100 includes a substance administration assembly 20. The substance administration assembly 20 is operable to store and deliver a substance such as but not limited to an antidote. The substance administration assembly 20 includes a syringe member 22 wherein the syringe member 22 is cylindrical in shape having an interior volume configured to receive and retain a substance. The syringe member 22 is mounted within the interior volume of the housing 10 so as to inhibit movement thereof. Operably coupled to the syringe member 22 is the plunger 24. Plunger 24 is movably coupled with the syringe member 22 wherein the plunger 24 is operable to traverse through the interior of the syringe member 22 in order to facilitate the removal of a substance disposed therein out of the syringe member 22 into the spring needle assembly 30 via tubing 27.

The plunger 24 is operably coupled to the plunger driver 32 wherein the plunger driver 32 is configured to provide movement of the plunger towards the syringe member 22. The plunger driver 32 includes upper portion 33 that is configured to couple to the top 34 of the plunger 24. The plunger driver 32 includes a lower portion 35 wherein the lower portion 35 includes teeth 36 formed thereon. The teeth 36 are formed so as to operably couple with the gear 39 driven by motor 40. Motor 40 is operable to rotatably move the gear 39 wherein the rotational movement of the gear 39 moves the plunger driver 32 so as to facilitate the transfer of material disposed in plunger 24 to the spring needle assembly 30 via tubing 27. It should be understood within the scope of the present invention that the motor 40 could be various types of electric motors such as but not limited to a servo motor. Additionally, it should be understood within the scope of the present invention that the gear 39 could be rotated at alternate revolutions per minute in order to deliver the substance disposed in the plunger 24 at alternate rates. It is further contemplated within the scope of the present invention that an alternate driving mechanism utilizing a spring loaded syringe could be employed. By way of example but not limitation, a syringe/needle assembly could be employed that is controlled by a servo motor/switch mechanism.

The spring needle assembly 30 is operable to inject the needle 51 into a wearer of the substance overdose detection apparatus 100. The spring needle assembly 30 is operably coupled to the central processing unit 15 and is configured to insert the needle 51 into the wearer subsequent receipt of a signal of a rapid drop in blood oxygen from the blood oxygen sensor 70. The spring needle assembly 30 is positioned in the housing 10 so as to be adjacent the wearer and allow the needle 51 to be journaled through a small aperture therein(not illustrated herein) so as to penetrate the wearer's subcutaneous or intramuscular level. It should be understood within the scope of the present invention that the spring needle assembly 30 could be placed in alternate locations such as but not limited to on the exterior of the housing 10 to facilitate needle 51 exchange. Furthermore, it should be understood within the scope of the present invention that the spring needle assembly 30 could employ more than one needle. The spring needle assembly 30 employs a spring 50 that is operably coupled to a lever switch 53. The lever switch 53 is configured to maintain the spring 50 in a biased compressed position. Ensuing receipt of a signal from the central processing unit 15, the lever switch 53 will move to its second position wherein the spring 50 will be released and as such facilitate the injection of the needle 51 into the wearer. While a spring needle assembly 30 has been illustrated herein, it should be understood within the scope of the present invention that the substance overdose detection apparatus 100 could employ alternate elements in order to facilitate the control and injection of a needle into a wearer of the substance overdose detection apparatus 100. By way of example but not limitation, an insertable cannula could be employed to deliver a substance to a wearer of the substance overdose detection apparatus 100.

The substance overdose detection apparatus 100 further includes a blood oxygen sensor 70. As is known in the art, blood oxygen can be measure indirectly by light absorption through a person's pulse. When an individual is experiencing an overdose of a substance such as but not limited to opioids, the individual will experience a rapid drop in blood oxygen levels. Detection of this drop and subsequent administration of an antidote by the substance overdose detection apparatus 100 can be employed in order to potentially save an individuals life. The blood oxygen sensor 70 is a conventional blood oxygen sensor and is operably coupled to the central processing unit 15 and transmits blood oxygen levels at routine intervals thereto when a wearer is using the substance overdose detection apparatus 100. It should be understood within the scope of the present invention that while the blood oxygen sensor 70 is illustrated in the housing 10 herein, that the blood oxygen sensor 70 could be a separate module to be secured on a body part such as but not limited to a finger. In the immediately aforementioned embodiment, the remote module would be wirelessly coupled or hardwired to the central processing unit 15. While a blood oxygen sensor 70 is employed in the preferred embodiment of the substance overdose detection apparatus 100, it is further contemplated within the scope of the present invention that the substance overdose detection apparatus 100 could utilize additional biometric sensors in place of and/or in conjunction with blood oxygen sensor 70. By of example but not limitation, the substance overdose detection apparatus 100 could measure heart rate and respiratory rate.

The substance overdose detection apparatus 100 further includes an audio alarm 75 and transceiver 80 both being operably coupled to the central processing unit 15. The audio alarm 75 is a conventional audio alarm that emits an audible signal in the event of administration of a substance by the substance overdose detection apparatus 100. This provides the ability to alert those near an individual wearing the substance overdose detection apparatus 100 that assistance may be needed. Additionally, the transceiver 80 can be programmed to transmit an emergency signal to either a caretaker or emergency personnel. The transceiver 80 is a conventional transceiver that is operable to employ either cellular or wireless communication protocols to transmit a signal to one or more parties. The substance overdose detection apparatus 100 includes a conventional power supply 90 wherein the power supply 90 is operable to provide the necessary electrical power for the substance overdose detection apparatus 100. Lights 95 are mounted into the housing 10 so as to be visible from the exterior thereof. The lights 95 are conventional LED lights that are utilized to provide status indication of parameters such as but not limited to injection status, power status or fault indication.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A drug overdose detection apparatus that is configured to be worn by a user and administer a substance to counteract a detected drug overdose wherein the substance overdose detection apparatus comprises:
a housing, said housing being configured to be worn by the user, said housing having an interior volume;
a central processing unit, said central processing unit having electronics to receive, store, transmit and manipulate data, said central processing unit operable to control operation of the drug overdose detection apparatus;
at least one biometric sensor, said at least one biometric sensor configured to detect, measure and record a biometric parameter of the user, said at least one biometric sensor operably coupled to said central processing unit;
a substance administration assembly, said substance administration assembly being disposed within the interior volume of the housing, said substance administration assembly configured to receive and retain a substance operable to counteract a drug overdose, said substance administration assembly operably coupled to said central processing unit; and
an injection member, said injection member operably coupled to said substance administration assembly, said injection member configured to receive the substance from the substance administration assembly and inject into the user.

2. The drug overdose detection apparatus as recited in claim 1, wherein said at least one biometric sensor is operable to measure a blood oxygen level of the user.

3. The drug overdose detection apparatus as recited in claim 1, wherein said at least one biometric sensor transmits a signal to the central processing unit upon detecting a change in a biometric parameter.

4. The drug overdose detection apparatus as recited in claim 3, and further including a transceiver, said transceiver being operably coupled to said central processing unit, said transceiver operable to transmit a signal to a third party subsequent administration of the substance to the user.

5. The drug overdose detection apparatus as recited in claim 4, and further including an audio alarm, said audio alarm operable to emit an audible signal ensuing administration of the substance to the user.

6. The drug overdose detection apparatus as recited in claim 5, wherein the substance administration assembly is configured to administer the substance at least once.

7. A drug overdose detection apparatus that is configured to be worn by a user and administer a substance to counteract a detected drug overdose wherein the substance overdose detection apparatus comprises:
a housing, said housing being configured to be worn by the user, said housing having an interior volume;
a central processing unit, said central processing unit having electronics to receive, store, transmit and manipulate data, said central processing unit operable to control operation of the drug overdose detection apparatus;
a blood oxygen sensor, said blood oxygen sensor configured to detect, measure and record blood oxygen of the user, said blood oxygen sensor operably coupled to said central processing unit;
a substance administration assembly, said substance administration assembly being disposed within the interior volume of the housing, said substance administration having a syringe member, said syringe member configured to receive and retain the substance operable to counteract a drug overdose, said syringe member further having a plunger member operably coupled thereto;
a spring needle assembly, said spring needle assembly further including a spring and at least one needle, said spring needle assembly operably coupled to said substance administration assembly, said spring needle assembly configured to inject the at least one needle into the user; and
wherein the drug overdose detection apparatus is operable to inject the substance ensuing detection of a drop in blood oxygen level by the blood oxygen sensor.

8. The drug overdose detection apparatus as recited in claim 7, wherein the substance administration assembly further includes a plunger driver, said plunger driver operably coupled to an upper end of the plunger member, said plunger driver having an upper portion and a lower portion.

9. The drug overdose detection apparatus as recited in claim 8, wherein the lower portion of the plunger driver has a plurality of teeth formed thereon.

10. The drug overdose detection apparatus as recited in claim 9, and further including a motor, said motor having a gear operably coupled thereto, said gear being operably coupled to said lower portion of said plunger driver, wherein rotation of said gear is operable to provide movement of the plunger driver.

11. The drug overdose detection apparatus as recited in claim 10, wherein the spring needle assembly further includes a lever switch, said lever switch operable to release tension from spring so as to inject the at least one needle into the user.

12. The drug overdose detection apparatus as recited in claim 11, and further including a transceiver, said transceiver being operably coupled to said central processing unit, said transceiver operable to transmit a signal to a third party subsequent administration of the substance to the user.

13. The drug overdose detection apparatus as recited in claim 12, wherein the blood oxygen sensor can be integrated into the housing or a remote module communicably coupled to the central processing unit.

14. The drug overdose detection apparatus as recited in claim 13, and further including an audio alarm, said audio alarm operable to emit an audible signal ensuing administration of the substance to the user.
